(19) United States
(12) Patent Application Publication
(10) Pub. No.: US 2024/0285922 A1
(43) Pub. Date: Aug. 29, 2024

(54) DEVICES, SYSTEMS, AND METHODS FOR ACCESS AND FLUID CIRCULATION WITHIN A BODY

(71) Applicant: BOSTON SCIENTIFIC SCIMED, INC., MAPLE GROVE, MN (US)

(72) Inventor: Michael S H Chu, Brookline, MA (US)

(73) Assignee: BOSTON SCIENTIFIC SCIMED, INC., MAPLE GROVE, MN (US)

(21) Appl. No.: 18/586,976

(22) Filed: Feb. 26, 2024

Related U.S. Application Data
(60) Provisional application No. 63/448,512, filed on Feb. 27, 2023.

(51) Int. Cl. A61M 39/02; A61M 25/01; A61M 25/09

(52) U.S. Cl. CPC A61M 39/0247; A61M 25/0102; A61M 25/09; A61M 2039/0273; A61M 2039/0279; A61M 2205/0266

(57) ABSTRACT

A first tubular medical device having a lumen through which a guidewire is insertable and over which the first tubular medical device may be guided to a target site within a patient. The proximal end of the guidewire may be extended into a lumen defined through a second medical device so the second medical device may be guided over the guidewire alongside the first tubular medical device to the target site. The guidewire may be extended into a distal opening in the first tubular medical device and out a side opening of the first tubular medical device to be available for guiding the second medical device alongside the first tubular medical device.

### CROSS REFERENCE TO RELATED APPLICATIONS

This application claims the benefit of priority under 35 U.S.C. § 119 to U.S. Provisional Application No. 63/448,512, filed Feb. 27, 2023, the entire disclosure of which is hereby incorporated by reference herein for all purposes.

### FIELD

The present disclosure relates generally to the field of medical devices, systems, and methods. In particular, the present disclosure relates to medical devices, systems, and methods for accessing a target site within a body and/or circulating a fluid within a body at a target site.

### BACKGROUND

Various medical procedures performed within a patient's body utilize one or more tubular elements extended into a natural orifice or a very small incision to access a treatment/target site within the patient's body (in contrast with open surgery involving larger cuts through which the target site within the patient's body is accessed more directly). Such tubular elements define at least one lumen therethrough through which devices, tools, instruments, etc., may be delivered to the target site. Additionally or alternatively, fluids may be delivered through the at least one lumen of the flexible tubular elements (such as to provide irrigation at the target site) and/or suction may be applied through the at least one lumen (such s to aspirate the target site, such as to remove materials therefrom). One example of a flexible tubular element configured to access a target site is a ureteral access sheath. The access sheath may be advanced to the target site with a dilator extended therein to facilitate advancement of the access sheath within the body. The dilator typically is removed once the distal end of the access sheath is positioned at the target site so that the lumen within the access sheath may be used for other purposes, such as instrument delivery, irrigation, aspiration, etc. A guidewire may be extended through the access sheath to facilitate delivery of additional devices to the target site (over the guidewire). Current access sheaths are limited by having a single lumen. Likewise, further devices, such as endoscopes, ureteroscopes, etc., extended through the access sheath are also limited to a single working channel therein. There is an ongoing need to lower/evacuate elevated temperatures at a target site (such as temperatures generated by laser energy delivered to the target site), high fluid pressure at the target site, and medical debris created by a procedure at the target site (e.g., stone fragments created during lithotripsy within a kidney), such as by application of negative pressure aspiration. There is also an ongoing need to provide irrigation (e.g., saline, contrast fluid, etc., such as to facilitate viewing, to flush the target site, to replenish aspirated fluids, to prevent organ (e.g., kidney) collapse, etc.). However, it is generally desirable to maintain a small profile for devices extended into the body, particularly devices extended through delicate passageways such as formed by a ureter. Whereas a single lumen device (e.g., a single working channel flexible ureteroscope) would lack a second channel to allow continuous fluid flow both in and out of the target site, a device with more than one lumen may have an undesirably wide profile. Various further challenges, such as navigating through curved internal passages/lumens, separating fluid flows between lumens within the patient, and other additional challenges must be addressed. There remains a need for low profile devices, systems, and methods for accessing a target site with more than one lumen. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system, for accessing a target site within the body of a patient, includes a first tubular medical device having a distal end, a proximal end, and a lumen defined therethrough between the distal end and the proximal end, the lumen configured to accept a guidewire to advance the first tubular medical device over the guidewire to the target site; and a second medical device advanceable alongside the first tubular medical device over the guidewire the first tubular medical device is advanced over, the guidewire extending through the lumen defined through the first tubular medical device and a lumen defined through the second medical device.

In some aspects, the first tubular medical device includes a tubular elongate body extending between the proximal end and the distal end of the first tubular medical device and a wall defining the lumen; the tubular elongate body defines a distal opening at the distal end of the first tubular medical device; the tubular elongate body defines a side opening through the wall thereof; and a guidewire is extendable into the distal opening of the tubular elongate body, through a distal portion of the lumen therein, and out the side opening through the wall of the tubular elongate body to be accessible for extending into the lumen defined through the second medical device. The side opening may be selected from any of a slot, a slit, a skive, a hole, or an aperture. In some aspects, the second medical device has a proximal end and a distal end, and includes a tubular elongate body extending between the proximal end and the distal end thereof having a wall defining a lumen configured to accept the portion of the guidewire extending out the side opening of the first tubular medical device. In some aspects, the tubular elongate body of the second medical device defines a distal opening at the distal end of the second medical device, the guidewire being extendable therethrough into the lumen defined through the second medical device. In some aspects, the second medical device is configured to allow the proximal end of the guidewire inserted therethrough to exit the system through the proximal end of the second medical device.

Optionally, the system further includes a cannula having a distal end and a proximal end. In some aspects, the cannula is configured to be insertable through the lumen defined through the first tubular medical device; the cannula has a wall defining a lumen therethrough extending between the distal end and the proximal end of the cannula; a distal opening is defined in the distal end of the cannula in communication with the lumen through the cannula; a side opening is defined in the cannula wall in communication with the lumen of the cannula; and the distance between the distal opening and the side opening of the tubular elongate body of the first tubular medical device is substantially the same as the distance between the distal opening and the side opening of the cannula.

Optionally, the first tubular medical device of the system is a sheath. Optionally, the system further includes a cannula insertable through the lumen defined through the first tubular medical device. Optionally, the system further includes a stylet insertable through a lumen defined through the cannula. Optionally, the system further includes a stylet insertable through the lumen defined through the first tubular medical device.

Optionally, the second medical device is any of a scope, a flexible scope, a steerable catheter, a guide catheter, a non-steerable catheter, a sheath, or an access sheath.

Optionally, the first tubular medical device includes a curved distal section formed of a shape memory material and capable of being straightened for insertion to the target site within the patient.

Optionally, the second medical device is steerable and capable of steering the first tubular medical device when the second medical device and the first tubular medical device are extended over a common guidewire.

In accordance with various principles of the present disclosure, a kit, for accessing a target site within the body of a patient, includes a guidewire; a first tubular medical device having a distal end, a proximal end, and a lumen defined therethrough between the distal end and the proximal end, the lumen configured to accept the guidewire and to be advanced over the guidewire to the target site; and a second medical device advanceable over the guidewire alongside the first tubular medical device with the guidewire extending through the lumen defined through the first tubular medical device and a lumen defined through the second medical device.

Optionally, the first tubular medical device includes a tubular elongate body extending between the proximal end and the distal end of the first tubular medical device and defining the lumen. Optionally, the tubular elongate body defines a distal opening at the distal end of the first tubular medical device. Optionally, the tubular elongate body defines a side opening through the wall defining the lumen. Optionally, the guidewire is extendable into the distal opening of the tubular elongate body, through a distal portion of the lumen therein, and out the side opening through the wall of the tubular elongate body to be accessible for extending into the lumen defined through the second medical device.

Optionally, the second medical device is any of a scope, a steerable catheter, a guide catheter, a non-steerable catheter, a sheath, or an access sheath.

In accordance with various principles of the present disclosure, a method of accessing a target site within a patient includes guiding a first tubular medical device over a guidewire; and guiding a second medical device over the same guidewire the first tubular medical device is guided and alongside the first tubular medical device.

In some aspects, the method further includes extending a proximal end of the guidewire into a distal end of the first tubular medical device and into a lumen extending therethrough, and out a side opening through a wall defining the lumen. In some aspects, the method further includes extending the proximal end of the guidewire into a distal end of the second medical device and into a lumen defined through the second medical device to guide the second medical device over the guidewire alongside the first tubular medical device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary.

For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a tubular medical device formed in accordance with aspects of the present disclosure.

FIG. 1A illustrates a detail view of detail 1A of FIG. 1.

FIG. 2 illustrates an exploded view of the tubular medical device illustrated in FIG. 1.

FIG. 3 illustrates a perspective view of a tubular medical device as in FIG. 1 mounted over a guidewire.

FIG. 4 illustrates an enlarged elevational view of the distal portion of the tubular medical device of FIG. 3.

FIG. 5 illustrates a perspective view of a first example of an embodiment of a second medical device inserted over the guidewire over which an example of an embodiment of a tubular medical device is inserted.

FIG. 6 illustrates a perspective view of a second example of an embodiment of a second medical device inserted over the guidewire over which an example of an embodiment of a tubular medical device is inserted.

FIG. 7 illustrates a perspective view of a third example of an embodiment of a second medical device inserted over the guidewire over which an example of an embodiment of a tubular medical device is inserted.

FIG. 8 illustrates an example of an embodiment of a use of a second medical device and first medical device such as illustrated in FIG. 7.

FIG. 8A illustrates detail 8A of FIG. 8.

FIG. 9 illustrates a perspective view of a second example of an embodiment of a tubular medical device formed in accordance with various principles of the present disclosure and along which a second medical device may be inserted in accordance with various principles of the present disclosure.

FIG. 10 illustrates an elevational view of the tubular medical device illustrated in FIG. 9.

FIG. 11 illustrates a perspective view of a third example of an embodiment of a tubular medical device insertable formed in accordance with various principles of the present disclosure and along which a second medical device may be inserted in accordance with various principles of the present disclosure.

FIG. 12 illustrates a side elevational view of the medical device illustrated in FIG. 11.

FIG. 13 illustrates an example of an embodiment of a cannula usable in a tubular medical device as disclosed herein.

FIG. 14 illustrates a perspective view of a fourth example of an embodiment of a second medical device inserted over the guidewire over which an example of an embodiment of a tubular medical device is inserted.

FIG. 15 illustrates a perspective view of a fourth example of an embodiment of a tubular medical device formed in accordance with various principles of the present disclosure and along which a second medical device may be inserted in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

In accordance with various principles of the present disclosure, a system with separate tubular medical devices is provided to perform a minimally-invasive (in contrast with open surgical) medical procedure within a patient's body. The separate tubular medical devices are operable in association with each other, as well as separable from each other for independent operation of each. In some aspects, a first tubular medical device is insertable into a patient's body over a guidewire, and a second medical device is insertable over the same guidewire into the patient's body. The distal ends of the first and second devices are guided, over the guidewire, to a target site within the patient's body. The first tubular medical device has a lumen defined therethrough. The lumen may be defined through a tubular elongate body of the first tubular medical device, reference being made alternately herein to the lumen of the first tubular medical device or of the tubular elongate body of the first tubular medical device without intent to limit. The second medical device typically is tubular as well, or at least has a lumen defined therein through which the guidewire extends for guiding the second device to the target site. The second medical device may have a tubular elongate body through which the lumen is defined. Accordingly, reference may be made alternately herein to the lumen of the second medical device or of the tubular elongate body of the second medical device without intent to limit. The guidewire may have properties selected based on the procedure to be performed and/or the nature of the anatomical site at which the target site is located and/or the devices being used therewith, and/or for other features or properties as may be appreciated by those of ordinary skill in the art.

More particularly, the first tubular medical device may be a mono-rail type tubular device such as a mono-rail catheter. The guidewire is extended into an opening at the distal end of the first tubular medical device and out a side opening defined in the wall of the first tubular medical device proximal to the distal opening. The first tubular medical device may be guided over the guidewire to a target site within a patient. The guidewire over which the first tubular medical device is guided to the target site may be considered to become a rail for additional devices inserted outside and alongside the outside of the first tubular medical device. Thus, a second medical device may be guided over the guidewire to the target site as well. The second medical device may be guided over the guidewire after the first tubular medical device has been guided to the target site. The guidewire may extend from a distal opening in the first tubular medical device out an opening in the wall of the first tubular medical device, or may traverse the entire length of the first tubular medical device.

The provision of a first tubular medical device configured to be guided over a guidewire and to position such guidewire for an additional device to be guided over such guidewire allows ready insertion and guiding of two separate devices, which may provide two separate lumens, to a target site. The devices can be used in conjunction with each other to provide continuous circulation of fluids in a treatment/collection system. For instance, the direction of fluid flow in the first tubular medical device may be in the opposite direction of the direction of fluid flow in the second medical device. Alternatively, the direction of fluid flow through the devices may be the same, with fluid flowing through one of the medical devices supplementing the flow of fluid through the other of the medical devices. In contrast with prior art devices, devices and systems formed in accordance with various principles of the present disclosure allow for simultaneous aspiration and irrigation at a target site. As may be appreciated, the devices may each have a smaller profile than the profile of a dual-lumen device, and thus each may be easier (than a dual-lumen device) to navigate within the patient's body to a target site. However, the lumen provided by at least one of the devices may be larger than a corresponding passage provided in prior devices (e.g., an irrigation lumen of the present disclosure is larger than irrigation lumens provided by prior devices and systems). Provision of more than one tubular medical device allows the medical professional greater freedom and flexibility and further options in determining the appropriate tubular medical device to use for irrigation and the appropriate tubular medical device to use for aspiration during a procedure, without requiring removal, exchange, etc., of devices. For instance, if desired, aspiration may be separated from an imaging device (e.g., to protect the imaging device from aspirated debris) by providing a tubular medical device for imaging, and a separate and independent tubular medical devices for aspiration. Moreover, each device is separately and independently navigated to the same target site, and may be separately and independently navigable, thereby facilitating delivery of each device to the target site. And, such separate and independent delivery allows for positioning of the distal ends of the tubular medical devices at different, spaced apart locations (e.g., axially or laterally with respect to each other) the delivery of irrigation fluid at a location apart from a location at which suction is applied to aspirate in the area of the target site. For instance, delivery of fluid to a target site to a location spaced apart from the location at which aspiration is performed may facilitate viewing of the aspiration location by not delivering fluid directly at the aspiration location that may scatter smaller fragments at the target site.

The first tubular medical device may be an access catheter defining a lumen extending therethrough and through which a guidewire may be passed. Optionally, a stylet or obturator or dilator may be positioned within the lumen of the first tubular medical device, such as to facilitate guiding of the first tubular medical device to the target site in a manner known to those of ordinary skill in the art. The first tubular medical device may have a preset curve or other non-straight configuration which may be held in a different configuration by the stylet or obturator. The second medical device may be any of a variety of devices usable with the first tubular medical device, such as, without limitation, an active, steerable device such as a scope, a flexible scope, a ureteroscope, a steerable catheter, etc.; or a passive device such as a catheter, a guide catheter, a sheath, etc., which may be steered or guided by a steerable device. Optionally, the second medical device is configured to position the guidewire such that a third medical device may be delivered over the guidewire to the target site. As may be appreciated, in accordance with various principles of the present disclosure, a first tubular medical device (e.g., an access sheath), with the guidewire along which it is advanced, may thus be used not only on its own, but also as a side rail for additional, separate devices. The profile of the first tubular medical device, such as the inner and outer diameters thereof, optionally is reduced relative to prior access sheaths.

Devices, systems, and methods in accordance with various principles of the present disclosure may be advantageously used in conjunction with lithotripsy and/or renal stone dusting procedures. For instance, devices, systems, and methods disclosed herein allow the simultaneous circulation of fluid into and out of a target site, such as within a kidney. Such fluid flow allows kidney stones, stone fragments, and debris, as well as elevated temperatures and pressures to be transported via fluid out of the collection system (i.e., aspirated out of the kidney) during lasing of the stones. Aspiration through the lumen of the first device and/or the second device may be by gravity, and/or by electro-mechanical mean such as a vacuum pump and/or an infusion pump, and/or by an electromechanical fluid management system that can sense pressure and/or temperature. The first tubular medical device may thus be an aspiration catheter, such as configured to aspirate stones (renal calculi, nephrolithiasis, or urolithiasis), stone fragments, residual stone dust, and/or other undesired particles out of a kidney, as well as to reduce temperature at the target site within the kidney by aspirating out fluids with elevated temperatures. While one device lumen may be used to aspirate a target site, the other device lumen may be used to replenish the aspirated-out fluid with, e.g., cool fluid to the target site, flushing fluids, treatment fluids, etc. The medical professional may choose which device lumen to use for aspiration and which to use for irrigation based on any of a variety of factors, such as the nature of the medical device with the lumen, the navigability of the medical device with the lumen, the size of the lumen, the amount of irrigation/aspiration to apply via the lumen, etc.

Various embodiments of devices, systems, and methods, such as for accessing a target site within a body and/or circulating fluid at a target site within a body, will now be described with reference to examples illustrated in the accompanying drawings. Reference in this specification to “one embodiment,”“an embodiment,”“some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,”“an embodiment,”“some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features in the drawings are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments of second medical devices are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

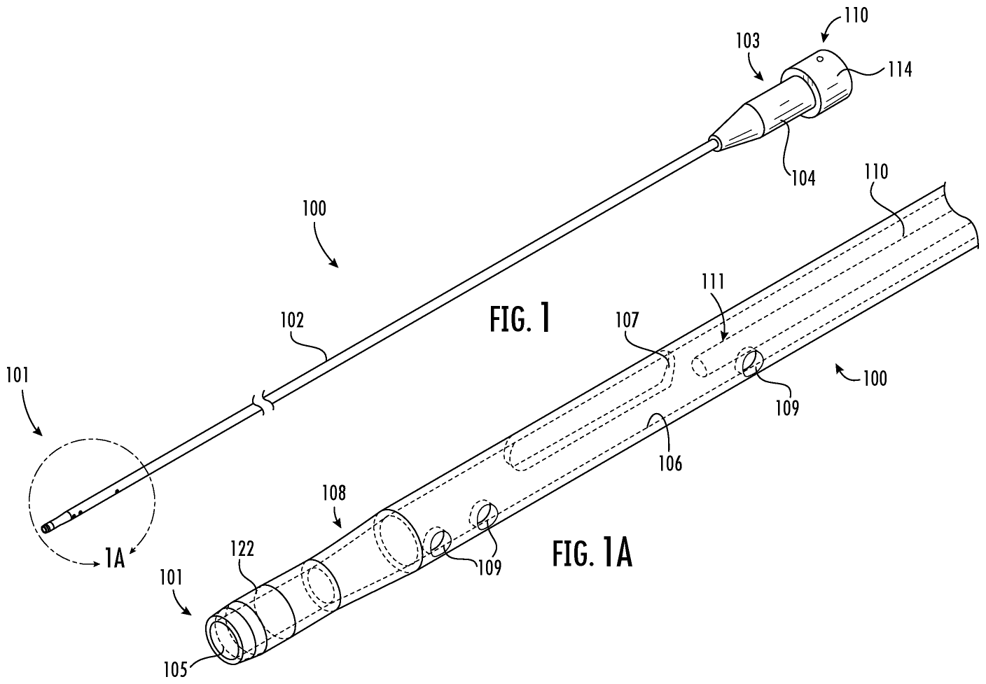

Turning now to the drawings, an example of an embodiment of a first tubular medical device 100 is illustrated in FIG. 1 as having a tubular elongate body 102 extending between a distal end 101 and a proximal end 103 of the first tubular medical device 100. The first tubular medical device 100 may be a catheter or other tubular device insertable into a patient, and typically is elongated and/or flexible. The distal end 101 of the first tubular medical device 100 thus may be configured for insertion to a target site within a patient's body. The proximal end 103 of the first tubular medical device 100 thus may be configured to extend outside the patient's body, such as to be accessed by a medical professional. In some embodiments, a hub 104 is provided at the proximal end 103 of the first tubular medical device 100. The hub 104 may be configured to be engageable by a medical professional, such as to guide the first tubular medical device 100 to the target site. Additionally or alternatively, the hub 104 may be configured to provide access to a lumen 106 (illustrated in FIG. 1A) defined through the tubular elongate body 102 and extending between the distal end 101 and the proximal end 103 of the first tubular medical device 100.

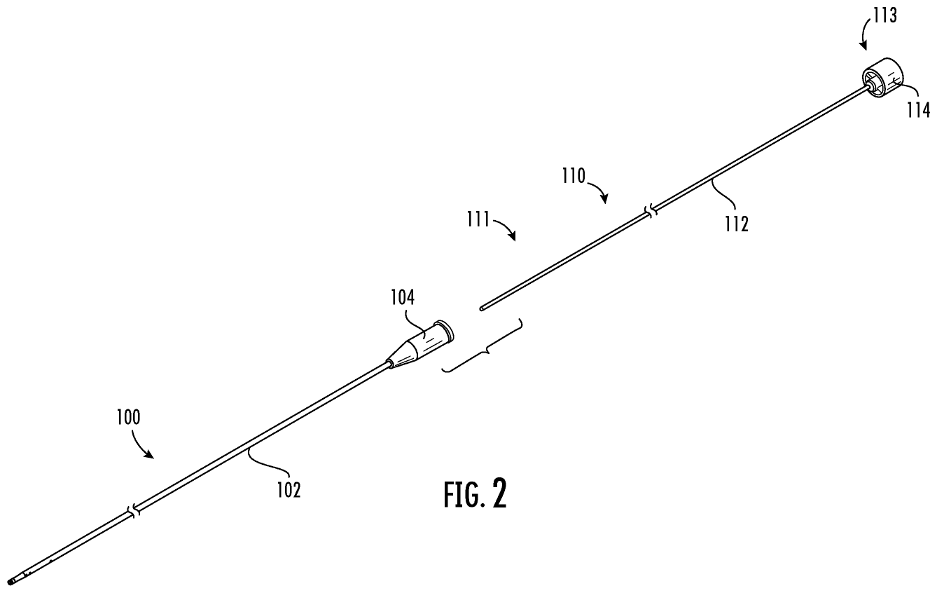

A stylet 110 is illustrated in FIG. 1 as positioned within the first tubular medical device 100. The stylet 110 is illustrated in greater detail in the exploded view of FIG. 2 as having an elongate body 112 extending between a distal end 111 and a proximal end 113 of the stylet 110. The distal end 111 of the stylet 110 is insertable into the lumen 106 of the first tubular medical device 100, such as via the hub 104 at the proximal end 103 of the first tubular medical device 100. The proximal end 113 of the stylet 110 typically remains outside and proximal to the first tubular medical device 100, such as to be accessible to a medical professional. The stylet 110 may have a cap 114 at the proximal end 113 thereof, configured to be grasped by a medical professional, such as to guide the stylet 110. The stylet 110 may be more rigid than the tubular elongate body 102 of the first tubular medical device 100 to facilitate pushability of the first tubular medical device 100 in a manner such as known to those of ordinary skill in the art.

In accordance with various principles of the present disclosure, the first tubular medical device 100 is configured to be inserted over a guidewire in a manner which permits additional tubular medical devices to be inserted over the same guidewire. An example of an embodiment of a distal end 101 of a first tubular medical device 100 which may be guided over a guidewire over which a second medical device may be guided, alongside and external to the first tubular medical device 100, is illustrated in FIG. 1A, showing detail 1A of FIG. 1. As may be appreciated with reference to FIG. 1A, the distal end 101 of the first tubular medical device 100 includes a distal opening 105 providing axial access to the lumen 106 extending generally longitudinally within the first tubular medical device 100. Additionally, a side opening 107 extending laterally through the wall of the tubular elongate body 102 of the first tubular medical device 100, provides lateral access to the lumen 106. The side opening 107 may have any of a variety of configurations, such as a slot (typically allowing a gradual guidewire exit angle from the lumen 106, such as in the example of an embodiment illustrated in FIG. 1A), a slit (e.g., a cut in the wall, such as by a sharp edge, which may be self-closing so that a guidewire is pushed therethrough), a skive, a hole (e.g., having a smaller cross-sectional area than a slot), an angled hole, an aperture, etc., which may be selected or determined based on the one or more devices to be used in conjunction with the first tubular medical device 100. As may be appreciated with reference to FIG. 1A, the distal end 111 of the stylet 110 may be positioned proximal to the side opening 107 so as not to interfere with access to the side opening 107. The first tubular medical device 100 may have a tapered distal section 108 transitioning the outer diameter of the first tubular medical device 100 (from the proximal end 103 thereof to the tapered distal section 108) to a smaller outer diameter at the distal end 101 of the first tubular medical device 100. The tapered distal section 108 may be formed to have a longitudinal extent, degree of taper, beginning and end diameters, etc., based on the target site into which the first tubular medical device 100 is to be inserted. For instance, the tapered distal section 108 may be configured to facilitate insertion of the first tubular medical device 100 into a patient's body (e.g., through a ureteral orifice). Optionally, a visualization marker 122, such as a radiopaque marker or band or filler material, may be provided adjacent the distal end 101 of the first tubular medical device 100 to facilitate visualization, navigation, placement, etc., of the first tubular medical device 100, such as under fluoroscopy. One or more additional side holes 109, each labeled with reference number 109 for the sake of convenience and without intent to limit, may be provided through the wall of the tubular elongate body 102 for various purposes, such as to allow flow of materials (e.g., solids or fluids) therethrough between the target site and the lumen 106 within the tubular elongate body 102. The additional side holes 109 may have any of a variety of configuration to facilitate use thereof. In some aspects, additional side holes 109 may be used for delivery or withdrawal of materials, such as during irrigation or aspiration. Additionally or alternatively, the side opening 107 may be used as an irrigation or aspiration port.

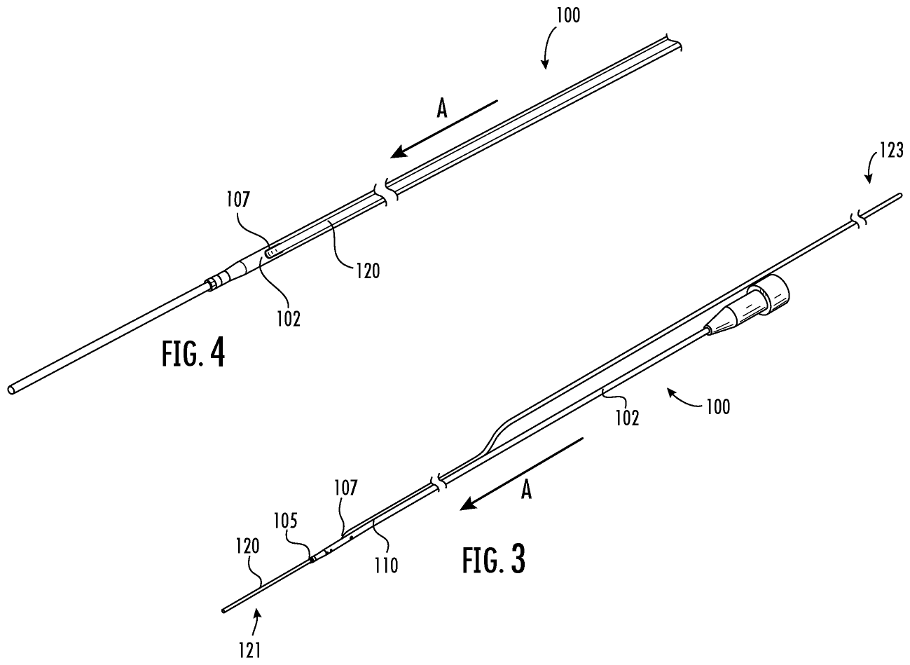

As illustrated in FIG. 3, a guidewire 120 is extendable through the lumen 106 of the first tubular medical device 100. For instance, a proximal end 123 of the guidewire 120 may be inserted into the distal opening 105 of the first tubular medical device 100, and the first tubular medical device 100 then advanced over the guidewire 120 in advancement direction A towards the distal end 121 of the guidewire 120. The proximal end 123 of the guidewire 120 is then extended laterally out the side opening 107 in the tubular elongate body 102 of the first tubular medical device 100, as illustrated in FIG. 3, to be available for insertion of another medical device thereover. The side opening 107 may be positioned proximal to the distal opening 105 of the first tubular medical device 100 a sufficient distance for the first tubular medical device 100 to be capable of being guided over the extent of the guidewire 120 extending from the distal opening 105 to the side opening 107 of the tubular elongate body 102. The distance of the side opening 107 from the distal opening 105 may also be selected/determined based on the interaction of the first tubular medical device 100 with a second medical device to be guided over the guidewire 120 (e.g., to facilitate steering of the second medical device with the assistance of the first tubular medical device 100 when both devices are mounted over a common guidewire 120, such as described in further detail below). The proximal end 123 of the guidewire 120 may be held outside the patient's body as the first tubular medical device 100 (optionally supported by a stylet 110) is advanced in the advancement direction A over the guidewire 120, into the patient's body, and to the target site within the patient's body. As may be appreciated with reference to FIG. 3 and FIG. 4, the guidewire 120 extends along the longitudinal extent of the tubular elongate body 102 as the first tubular medical device 100 is advanced in the advancement direction A.

As noted above, a second medical device may be inserted and guided over the same guidewire 120 that the first tubular medical device 100 is inserted and guided over to a target site within a patient. The second medical device may be any of a variety of medical devices, such as a tubular device, configured to be insertable alongside the first tubular medical device 100 and usable therewith in any of a variety of manners for any of a variety of medical procedures, such as within a patient's body.

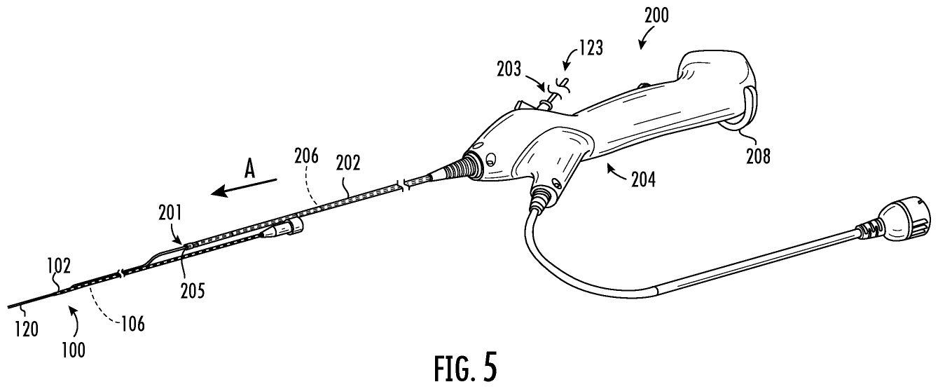

An example of an embodiment of a second medical device 200 insertable over a guidewire 120 over which a first tubular medical device 100 has been advanced is a steerable medical device, such as a medical scope as illustrated in FIG. 5. The example of an embodiment of a scope illustrated FIG. 5 is a ureteroscope, although other forms of scopes (e.g., endoscope, bronchoscope, duodenoscope, colonoscope, cholangioscope, arthroscope, cystoscope, hysteroscope, etc.) may be used instead. The second medical device 200 has a tubular elongate body 202 extending between a distal end 201 and a proximal end 203 of the second tubular medical device 200 and insertable into a patient's body to a target site therein. A handle 204 may be provided at the proximal end 203 of the second medical device 200 to facilitate steering of the second medical device 200, such as to navigate the tubular elongate body 202 to the target site, and/or to deliver materials to or remove materials from the target site via the tubular elongate body 202. A working channel 206 is defined through the tubular elongate body 202 and extends longitudinally therethrough, between the distal end 201 of the second medical device 200 and the proximal end 203 of the second medical device 200, such as for access thereto via the handle 204. In some embodiments, the second medical device 200 has a single working channel, although the present disclosure need not be so limited. The working channel 206 of the second medical device 200 is extended over the proximal end 123 of the guidewire 120 which extends out of the side opening 107 of the first tubular medical device 100. For instance, the proximal end 123 of the guidewire may be inserted into a distal opening 205 at the distal end 201 of the tubular elongate body 202 of the second medical device 200 in communication with the working channel 206 of the second medical device 200. The proximal end 123 of the guidewire 120 may be extended out the proximal end 203 of the second medical device 200, such as to be held external to the patient's body (e.g., along the handle 204 of the second medical device 200). The second medical device 200 is advanced over the guidewire 120 in the advancement direction A to the target site.

Once the respective distal ends 101, 201 of the first tubular medical device 100 and the second medical device 200 are at or near the target site, the second medical device 200 may be steered (e.g., with the assistance of the handle 204 at the proximal end 203 thereof). For instance, the second medical device 200 may have a deflectable distal end 201 steerable to a specific location within the patient's body. Additionally or alternatively, the deflectable distal end 201 of the second medical device 200 may also steer the first tubular medical device 100 to a specific location within the patient's body. For instance, the distal ends 101, 201 of the first tubular medical device 100 and the second medical device 200 may be steered to a renal stone to be treated, or to a target calyx (usually a mid-pole or upper-pole calyx for ease of access) into which renal stones may be repositioned to be treated. With the guidewire 120 still threaded through both devices 100, 200, the distal end 201 of the second medical device 200 is advanced toward the proximal end 101 of the first tubular medical device 100, to the side opening 107 of the first tubular medical device 100. The distal end 201 of the second medical device 200 is deflectable (such as by using a deflection knob 208 on the handle 204) towards the target site (e.g., a target calyx). Scope visualization and/or fluoroscopy may be used to guide movements of the second medical device 200 in a manner known to those of ordinary skill in the art. The deflection of the distal end 201 of the second medical device 200 may also deflect the guidewire 120 and the distal end 101 of the first tubular medical device 100 towards the target site. To enter the target site, the distal end 101 of the first tubular medical device 100 is advanced towards the target site over the guidewire 120. Once the distal end 101 of the first tubular medical device 100 is in the desired position, the stylet 110 (if provided) and the guidewire 120 may be removed. Removal of the guidewire 120 from the first tubular medical device 100 and from the second medical device 200 separates the devices 100, 200 to allow separate and independent operation thereof. For instance, the second medical device 200 may be maneuvered independently of the first tubular medical device 100 to perform other functions, while the first tubular medical device 100 may remain in the position at which it has been delivered to the target site without being disturbed by manipulation and/or movement of the second medical device 200. The removal of the guidewire 120 from the first tubular medical device 100 and from the second medical device 200 also frees the lumen 106 of the first tubular medical device 100 and the working channel 206 of the second medical device 200. Materials may thus be more readily transported through the devices 100, 200. Moreover, materials may be passed independently through the lumen 106 through the first tubular medical device 100 and the working channel 206 of the second medical device 200 to allow one of the lumen 106 or the working channel 206 to serve as an inlet to the target site and the other of the lumen 106 or the working channel 206 to serve as an outlet from the target site. For instance, fluid (e.g., saline for flushing the target site) may be delivered to the target site by one of the lumen 106 through the first tubular medical device 100 or the working channel 206 of the second medical device 200, while materials may be aspirated through the other of the lumen 106 through the first tubular medical device 100 or the working channel 206 of the second medical device 200. In another example, fluid (e.g., saline for flushing the target site) may be delivered to the target site by one of the lumen 106 through the first tubular medical device 100 or the working channel 206 of the second medical device 200, while fluid may be independently delivered to the target site by the other of the lumen 106 through the first tubular medical device 100 or the working channel 206 of the second medical device 200. Optionally, both the lumen 106 through the first tubular medical device 100 and the working channel 206 of the second medical device 200 may be used to aspirate. Typically, the larger of the lumens at the target site is used for aspiration to reduce the likelihood of clogging the aspiration lumen.

An example of a use of the system illustrated in FIG. 5, with a first tubular medical device 100 which may be in the form of an access catheter, and a second medical device 200 which may be in the form of a ureteroscope, is removal of stones from a patient's kidney. The target site may thus be a calyx. The optional stylet 110, and the guidewire 120 may be withdrawn from the lumens 106, 206 of the devices 100, 200 once the distal end 101 of the first tubular medical device 100 has reached the renal pelvis or otherwise is in proximity to the stone and/or at the target calyx. A device for treating (destroying, removing, etc., kidney stones) may be inserted through the working channel 206 of the tubular elongate body 102, such as to reposition a renal stone to a target calyx, or to perform laser lithotripsy with an inserted laser fiber. Larger stones can be laser fragmented to smaller or dust-sized fragments, and aspirated out of the body during or after laser lithotripsy. For instance, in a first embodiment, a laser fiber is inserted into the working channel 206 of the second medical device 200 to perform laser lithotripsy. The working channel 206 of the second medical device 200 may also be used as a fluid inlet channel and for flushing the target site, while the lumen 106 of the first tubular medical device 100 is used to aspirate/suction out fluid along with elevated heat, pressure, and debris such as stone debris. In a second embodiment, a laser fiber is inserted into the working channel 206 of the second medical device 200 to perform laser lithotripsy. The working channel 206 of the second medical device 200 may also be used as the fluid outlet channel to aspirate/suction out elevated fluid, heat, pressure, debris (such as stone debris), etc., while the first tubular medical device 100 is used as an inlet channel to replenish fluid to the target site. It will be appreciated that a system as disclosed herein, with separate devices 100, 200 inserted into the target site, allows for ready fluid flow in opposite directions, with the direction of fluid flow through the first tubular medical device 100 being opposite the direction of fluid flow through the second medical device 200. However, in some embodiments, fluid flow through one of the devices 100, 200 may supplement fluid flow through the other of the devices 100, 200, with the fluid flows being independently controllable to achieve the desired fluid flow regulation in a manner more readily achievable than with a single device. As may be appreciated, increased suction may be required at certain times during a procedure, such as to suction out heavier materials and/or to suction at a faster speed. Such increase in suction may also reduce the volume of fluid facilitating performance of the procedure at the target site. Distribution of an increase in irrigation flow to the target site across more than one lumen helps dissipate any increase in pressure which may otherwise accompany the increase in irrigation flow if limited to a single lumen.

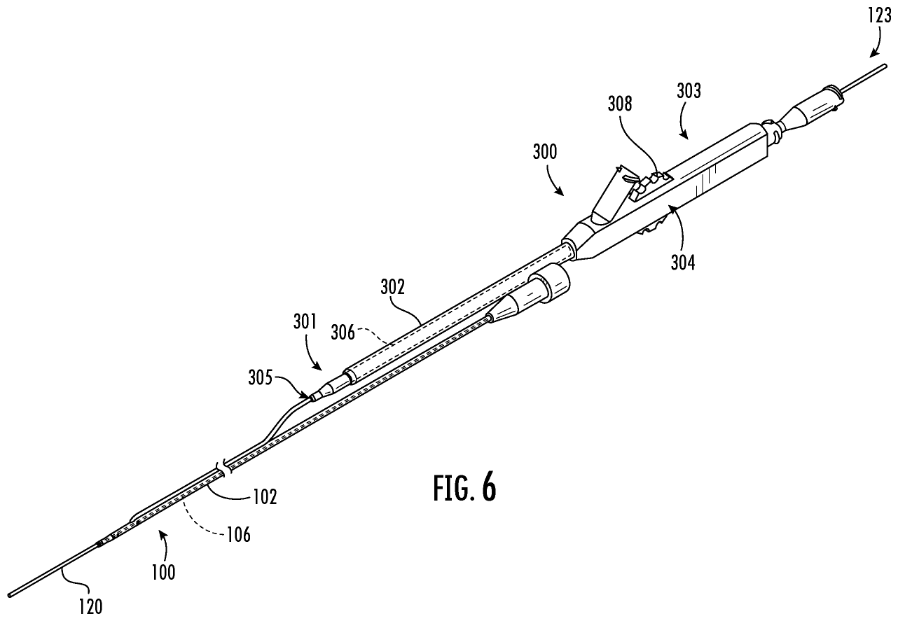

It will be appreciated that the second medical device need not provide direct visualization such as provided by a scope as in the example of an embodiment illustrated in FIG. 5. For instance, in the example of an embodiment of a system formed in accordance with various principles of the present disclosure and illustrated in FIG. 6, a second medical device 300 may be a steerable medical device guided over the same guidewire 120 over which the first tubular medical device 100 is guided to a target site within a patient. For instance, the example of an embodiment of a steerable medical device illustrated in FIG. 6 is a deflectable/steerable guide catheter. The second medical device 300 illustrated in FIG. 6 has a tubular elongate body 302 extending between a distal end 301 and a proximal end 303 of the second tubular medical device 300 and insertable into a patient's body to a target site therein. A handle 304 may be provided at the proximal end 303 of the second medical device 300 to facilitate steering of the second medical device 300 such as to navigate the tubular elongate body 302 to the target site, and/or to deliver materials to or remove materials from the target site via the tubular elongate body 302. For instance, the handle 304 may include a deflector knob 308 operably coupled with the tubular elongate body 302 to move or bend the tubular elongate body 302 in a direction to guide the distal end 301 of the second medical device 300 to the target site. The second medical device 300 has a lumen 306 extending longitudinally through the tubular elongate body 302, between the distal end 301 of the second medical device 300 and the proximal end 303 of the second medical device 300, such as for access thereto via the handle 304. In some embodiments, the second medical device 300 has a single lumen 306 therethrough, although the present disclosure need not be so limited. The lumen 306 of the second medical device 300 is extended over the proximal end 123 of the guidewire 120 which extends out of the side opening 107 of the first tubular medical device 100. For instance, the proximal end 123 of the guidewire may be inserted into a distal opening 305 at the distal end 301 of the tubular elongate body 302 of the second medical device 300 in communication with the lumen 306 of the second medical device 300. The proximal end 123 of the guidewire 120 is extended out the proximal end 303 of the second medical device 300, such as to be held external to the patient's body (e.g., along the handle 302 of the second medical device 300). The second medical device 300 is advanced over the guidewire 120 in the advancement direction A to the target site. The second medical device 300 may be advanced over the guidewire 120, alongside the first tubular medical device 100, to be placed alongside the first tubular medical device 100 at the target site.

As in the example of an embodiment illustrated in FIG. 5, by providing separate devices 100 and 300, separate lumens 106, 306 may allow greater functionality than achieved by prior art devices. For instance, the direction of fluid flow through the first tubular medical device 100 and the second medical device 200 may be in opposite directions, facilitating simultaneous irrigation and aspiration at the target site in a manner not readily achieved by prior art devices. Moreover, the lumen 306 of the second medical device 300 may be larger than the lumen 106 of the first tubular medical device 100. The larger lumen 306 of the second medical device 300 may thus advantageously be used as the outlet lumen of the system of the example of an embodiment illustrated in FIG. 6, such as for removing materials from the target site.

Similar to the example of an embodiment illustrated in FIG. 5, the example of an embodiment illustrated in FIG. 6 may be used for removal of kidney stones. For instance, the system illustrated in FIG. 6 may be used after laser lithotripsy, to aspirate stone fragments from the target site. A ureteroscope may be inserted over the guidewire 120 to perform the lithotripsy treatment. The ureteroscope may be removed after lithotripsy has been performed, leaving the guidewire 120 in place for guiding the second medical device 300 illustrated in FIG. 6 to the target site.

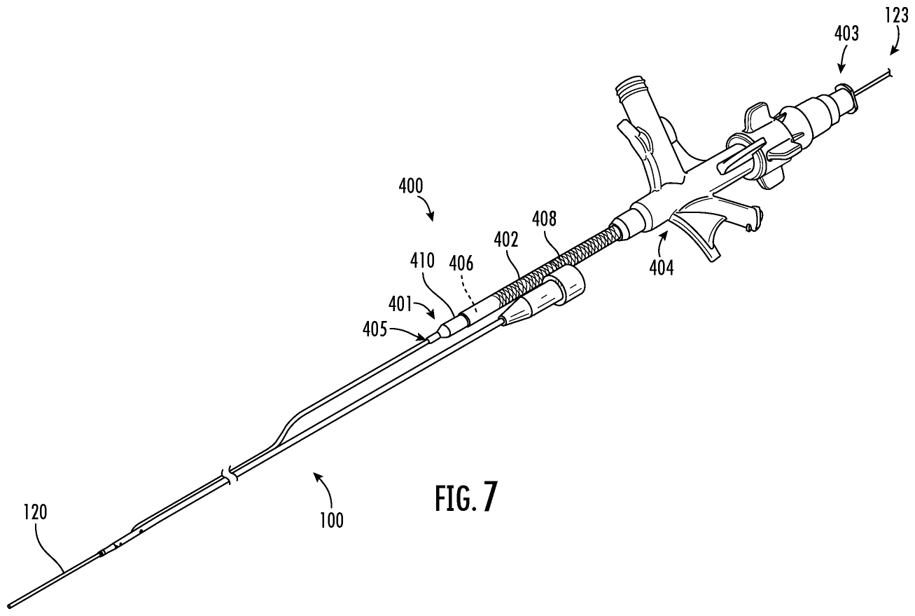

In contrast with the above-described examples of embodiments, a second medical device usable with a first tubular medical device 100 such as described above need not be steerable. For instance, as illustrated in FIG. 7, the second medical device 400 may be a passive sheath with a tubular elongate body 402 extending between a distal end 401 and a proximal end 403 of the second tubular medical device 400 and insertable into a patient's body to a target site therein. A lumen 406 is defined through the tubular elongate body 402, extending longitudinally between the distal end 401 and the proximal end 403 of the second medical device 400. As with the above-described examples of embodiments of second medical devices 200, 300, the example of an embodiment of a second medical device 400 illustrated in FIG. 7 is guided to a target site within a patient's body over the same guidewire 120 over which the first tubular medical device 100 is guided. As described above with reference to the second medical devices 200, 300, the proximal end 123 of the guidewire 120 may be inserted into a distal opening 405 at the distal end 401 of the tubular elongate body 402 of the second medical device 400, extended proximally through the lumen 406 of the second medical device 400, and out the proximal end 403 of the second medical device 400. The second medical device 400 may then be advanced over the guidewire 120, alongside the first tubular medical device 100, to the target site. Optionally, a dilator 410 is positioned within the lumen 406 of the tubular elongate body 402, such as to facilitate advancement of the second medical device 400 to the target site. When both devices 100, 400 have been delivered to or near the target site, the dilator 410 may be removed from the lumen 406 of the second medical device 400. Optionally, the guidewire 120 is also removed from the second medical device 400. A scope (such as illustrated in FIG. 5) may then be inserted into the lumen 406 of the second medical device 400 and advanced to the target site. In embodiments in which the guidewire 120 is left in place within the second medical device 400, the scope may be guided over the guidewire 120 into the tubular elongate body 402 of the second medical device 400. The scope may be steered (e.g., the distal end of the scope may be deflected) to guide and steer a flexible portion of the tubular elongate body 402 adjacent the distal end 401 of the second medical device 400. In some embodiments, a distal portion 408 of the tubular elongate body 402 of the second medical device 400 may be reinforced with coils and/or braided, such as illustrated and as known to those of ordinary skill in the art.

In the example of an embodiment of an access system illustrated in FIG. 7, the working channel of the scope inserted through the lumen 406 of the second medical device 400 may serve as the fluid inlet lumen to the target site, providing fluid for irrigating the target site. The outlet channel for the target site may be defined by the space between the lumen 406 of the second medical device 400 (the inner diameter of the tubular elongate body 402) and the outside of the scope. More specifically when the scope is inserted into the lumen 406, the space between the inner diameter of the lumen 406 and the outer diameter of the scope may define the outlet channel for the target site through which materials are aspirated. Such space typically has a larger cross-sectional area than the cross-sectional area of the scope. The lumen 106 through the first tubular medical device 100 may be used to supplement irrigation of the target site, such as to aid in maintaining a constant pressure or a constant fluid volume within an anatomical structure so that the anatomical structure is not over-pressurized or, in contrast, suctioned to the point of collapse. The lumen 106 through the first tubular medical device 100 can be formed with a large enough inner diameter to provide sufficient fluid to a target site independently, or to be used in conjunction with the working channel of a scope used at the target site. In some aspects, it may be preferable to irrigate only through the lumen 106 through the first tubular medical device 100 so as to avoid the possibility of irrigation fluid delivered through the scope's working channel being counteracted by suction applied through the space between the inner diameter of the lumen 406 and the outer diameter of the scope.

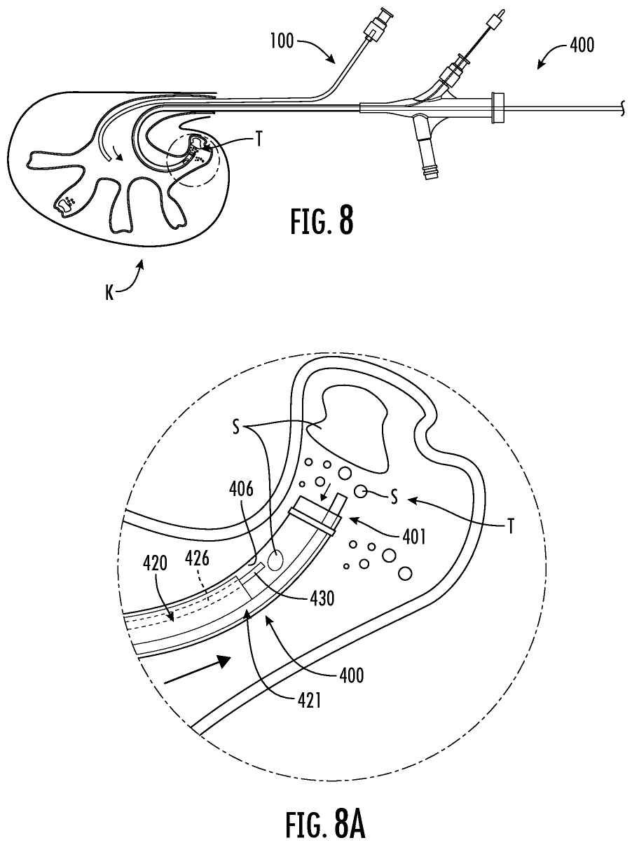

The example of an embodiment of a system illustrated in FIG. 7 may be used for laser lithotripsy, such as illustrated in FIG. 8. A flexible ureteroscope 420 may be inserted through the lumen 406 of the second medical device 400 to perform a lithotripsy procedure at the target site T within a kidney K. Optionally, as illustrated in the detail view of FIG. 8A, the distal end 421 of the ureteroscope 420 is withdrawn into the second medical device 400, and/or the second medical device 400 is advanced distal to the ureteroscope 420, to provide lasing from within the lumen 406 of the second medical device 400, and/or to suction/withdraw/remove larger stones against the distal end 421 of the ureteroscope 420 as the ureteroscope 420 is being withdrawn through the second medical device 400. As may be appreciated with reference to the detail view of FIG. 8A, a laser fiber 430 (e.g., any suitable energy-emitting device capable of breaking up kidney stones) may be extended through the working channel 406 of the ureteroscope 420 distal to the distal end 421 of the ureteroscope 420, yet within the lumen 406 of the second medical device 400. The laser fiber 430 may lase stones S at the target site or which have been suctioned into the lumen 406 of the second medical device 400 to stone dust. Stone dust created during lithotripsy may be aspirated through the space between the inner diameter of the lumen 406 of the second medical device 400 and the outer diameter of the ureteroscope 420. Optionally, irrigation from the working channel 406 of the ureteroscope 420 can be shut off or lowered such that the irrigation flow does not counteract the suction/aspiration flow between the inner diameter of the second medical device 400 and the outer diameter of the ureteroscope 420. Such suction will aspirate the “contained” dust, elevated temperature, and elevated pressure out of the lumen 406 of the second medical device 400 as the stone S is being lased. The first tubular medical device 100 can be positioned at a location spaced away from the stone S, such as illustrated in FIG. 8, to avoid the irrigation flow therefrom from scattering stone dust away from the second medical device 400. Distal flushing and irrigation can be provided by the working channel 406 of the ureteroscope 420 when, for instance, the ureteroscope 420 is positioned just proximal to the distal end 401 of the second medical device 400. Additionally or alternatively, the distal end 421 of the ureteroscope 420 may be extended distal to the distal end 401 of the second medical device 400 so that the laser fiber 430 may lase larger stones S stones to fit inside the second medical device 400.

The ureteroscope 420 optionally may be withdrawn so that the distal end 421 of the ureteroscope 420 is positioned within a proximal fitting 404 at the proximal end 403 of the second medical device 400. Such positioning of the ureteroscope would allow larger stone fragments to be aspirated out of the lumen 406 of the tubular elongate body 402 of the second medical device 400. To allow a larger sized stone or volume of stones to be aspirated out, without maintaining the tip of the ureteroscope tip at the proximal fitting 404, the ureteroscope may be removed entirely from the second medical device 400, and the entire lumen 406 of the second medical device 400 may then be used as the outlet lumen (e.g., for aspirating the target site). The first tubular medical device 100 may be used as the fluid inlet lumen (e.g., for irrigating the target site).

In another instance of use of a system as illustrated in FIG. 7, a large diameter device, such as a laser fiber or retrieval device, may be delivered through the inlet channel of a scope inserted through the second medical device 400. Such larger diameter device may predominantly block the inlet working channel of the scope, or may interfere with supply of fluid to (e.g., for irrigation of) the target site when the inlet volume demands/requires too high of an inlet pressure through the inlet working channel of the scope. In such instance, the first tubular medical device 100 may be used as the main source of inlet flow (e.g., for irrigation) or may supplement the inlet fluid flow of the scope working channel.

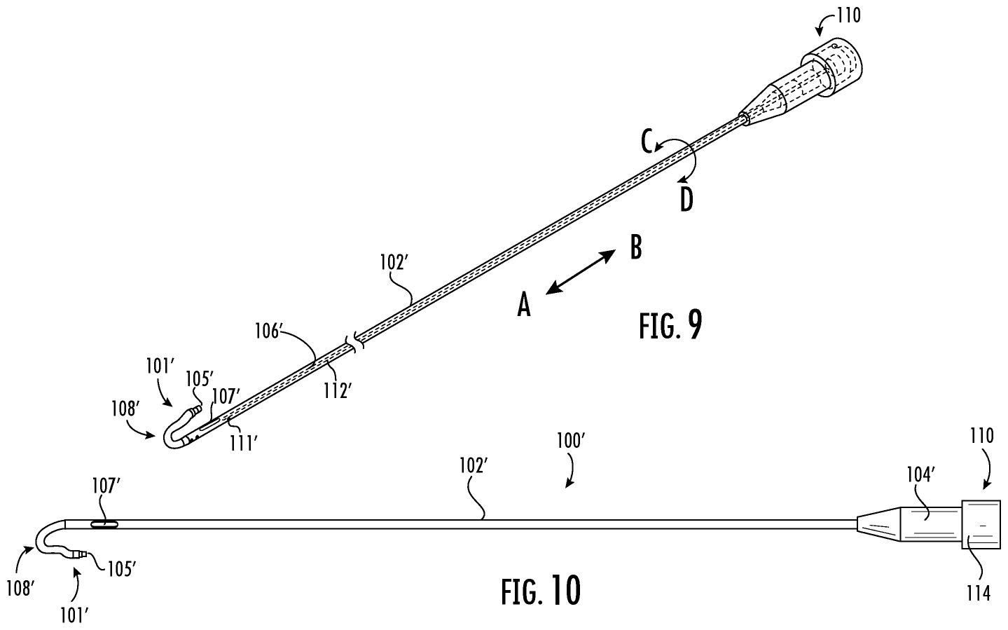

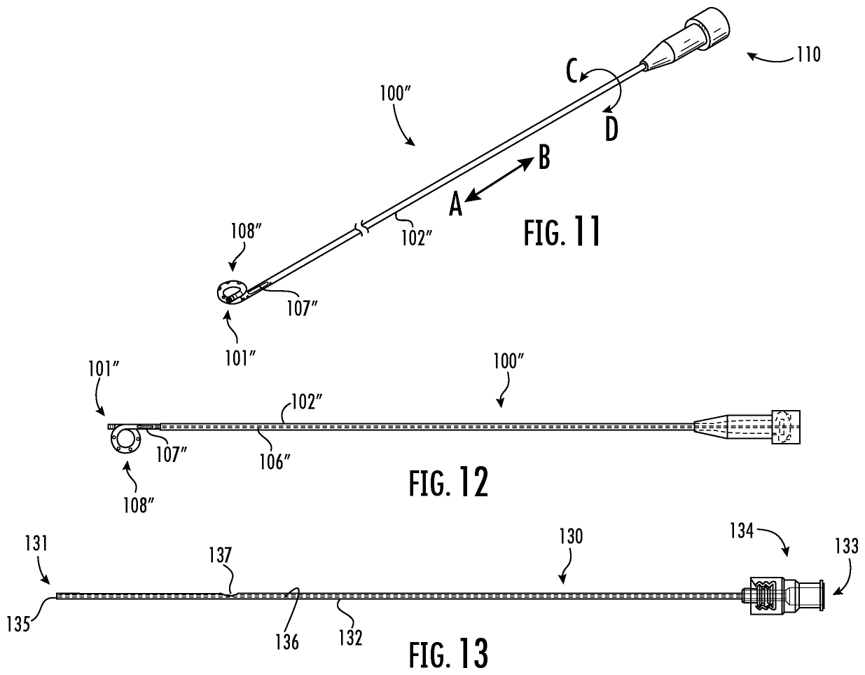

It will be appreciated that various modifications to a first tubular medical device as described herein may be made without departing from the various principles of the present disclosure. For instance, a portion (e.g., a distal portion) of the first tubular medical device may be formed into a pre-set, non-straight shape. For instance, at least a portion of the first tubular medical device may be formed of a shape memory material and curved to anchor the first tubular medical device at a target site. A stylet, obturator, dilator, cannula, guidewire, etc., may be inserted through a lumen defined through such first tubular medical device to maintain a generally straight configuration of the first tubular medical device to facilitate insertion and advancement to a target site within a patient's body. The first tubular medical device can be controlled (deflected, steered, etc.) and positioned as the stylet, obturator, dilator, cannula, guidewire, etc., is being withdrawn from the curved portion of the first tubular medical device. Partial withdrawal of the stylet, obturator, dilator, cannula, guidewire, etc., may cause the first tubular medical device to deflect from a generally straight configuration to another (e.g., curved) shape as the first tubular medical device returns to a pre-set shape. The stylet, obturator, dilator, cannula, guidewire, etc., may be removed once the distal end of the first tubular medical device has reached the target site to allow the shape memory material to return to its pre-set shape to anchor the first tubular medical device with respect to the target site. Examples of embodiments of a first tubular medical device with a curved distal section are illustrated in FIG. 9, FIG. 10, FIG. 11, and FIG. 12. The curved distal section may be formed of a material which may be heat set into a curve. Examples of suitable materials include any of a variety of biocompatible polymers such as nylon, rigid polyamide block and soft polyether block copolymers (e.g., Pebax®), polyester, polyethene, pharmaceutical grade thermoplastic elastomers, flexible polyvinylchloride (PVC), urethane, polyurethane, thermoplastic polyurethanes (TPU); or shape memory materials such as Nitinol, etc. The shape (e.g., curvature) of the distal section of the first tubular medical device may be based on the nature of the target site, such as to retain the first tubular medical device in place with respect to the target site and/or to position the distal end thereof in a desired/optimal orientation with respect to the target site. For instance, the distal section of a first tubular medical device formed in accordance with various principles of the present disclosure may be curved (e.g., heat set) to a bend of 0° (straight), 30°, 45°, 90°, 135°, 180°, 210°, 270°, 360° degrees (a pigtail), etc., including any angle therebetween.

In the example of an embodiment of a first tubular medical device 100′ illustrated in FIG. 9 and FIG. 10, the curved distal section 108′ has an approximately 180° bend, such as to reach the lower pole of the renal pelvis. In the example of an embodiment of a first tubular medical device 100″ illustrated in FIG. 11 and FIG. 12, the curved distal section 108′ has a pigtail configuration. The respective distal sections 108′, 108″ of the illustrated first tubular medical device 100′, 100″ may be heat set or otherwise formed to return to a curved or bent configuration after being held in a straightened configuration.

The respective curved distal sections 108′, 108″ of the examples of embodiments of first tubular medical devices 100′, 100″ illustrated in FIG. 9 and FIG. 10, and FIG. 11 and FIG. 12, respectively, may be held in a straightened configuration by a stylet, obturator, dilator, cannula, guidewire, etc., extending through a lumen defined through. The examples of embodiments of a first tubular medical device 100′, 100″ with a curved distal section 108′, 108″ are illustrated in FIG. 9 and FIG. 10, and FIG. 11 and FIG. 12, respectively, as supported by a stylet 110, such as described above with reference to FIG. 2. For instance, the distal end 111 of the stylet 110 may be positioned proximal to the side opening 107′, 107″ of the tubular elongate body 102 of the first tubular medical device 100′, 100″ so as not to interfere with access to the side opening 107′, 107″ in the respective tubular elongate body 102′, 102″ of the respective first tubular medical device 100′, 100″. The stylet 110 may extend proximally from to a cap 114 at the proximal end 113 of the stylet 110 positioned proximal to the proximal end 103′, 103″ of the first tubular medical device 100′, 100″. It is noted that the same reference characters as used in FIG. 1 and FIG. 2 are used to indicate substantially similar elements of the stylet 110 illustrated in FIGS. 9-12, reference being made to the above description of a stylet 110 for the sake of brevity and without intent to limit.

A guidewire (such as the above-described guidewire 120), which may be a super-stiff guidewire, can be threaded into the distal hole 105′, 105″ in the respective tubular elongate body 102′, 102″ of the respective first tubular medical device 100′, 100″, and out the respective side opening 107′, 107″ to straighten the curved distal section 108′, 108″ for delivery into the patient's body and to the target site. In some embodiments, such as illustrated in FIG. 9 and FIG. 11, the side opening 107′, 107″ is positioned alongside the curved distal section 108′, 108″ of the first tubular medical device 100′, 100″ (e.g., generally along the same position circumferentially around the first tubular medical device 100′, 100″). In some embodiments, such as illustrated in FIG. 10 and FIG. 12, the side opening 107′, 107″ is positioned spaced circumferentially apart from the curved distal section 108′, 108″ of the first tubular medical device 100′, 100″ (e.g., greater than approximately 5° apart, such as 90° apart, as illustrated in FIG. 10 and FIG. 12, or even 180° apart, including increments of 1° therebetween). As in the example of an embodiment illustrated in FIG. 1 and FIG. 1A, one or more additional side holes 109′, 109″ may be provided along the tubular elongate body 102′, 102″. If such additional side holes 109′, 109″ are located on the curved distal section 108′, 108″ of the first tubular medical device 100′, 100″, the additional side holes 109′, 109″ preferably are located on the sides or along the inner radius of the curved distal section 108′, 108″. As may be appreciated, holes along the outer radius of the curved distal section 108′, 108″ may allow a guidewire being threaded into the distal opening 105′, 105″ to protrude out from an additional side hole 109′, 109″ along the outer radius of the curved distal section 108′, 108″ as the guidewire is proximally advanced into the lumen 106′, 106″ in the respective tubular elongate body 102′, 102″ of the respective first tubular medical devices 100′, 100″, making the task of guiding the first tubular medical device 100′, 100″ over the guidewire more difficult.

When the guidewire or stylet 110 is withdrawn (proximally, and generally slowly as may be appreciated by those of ordinary skill in the art) from the first tubular medical device 100′, 100″, the curved distal section 108′, 108″ is able to return (generally slowly) to its curved pre-set (e.g., heat set) shape. The first tubular medical device 100′, 100″ may be moved axially (along direction A-B) or rotationally (along direction C-D), such as when the curved distal section 108′, 108″ has been returned to its curved shape, to position the curved distal section 108′, 108″ in a particular/desired location with respect to the target site.

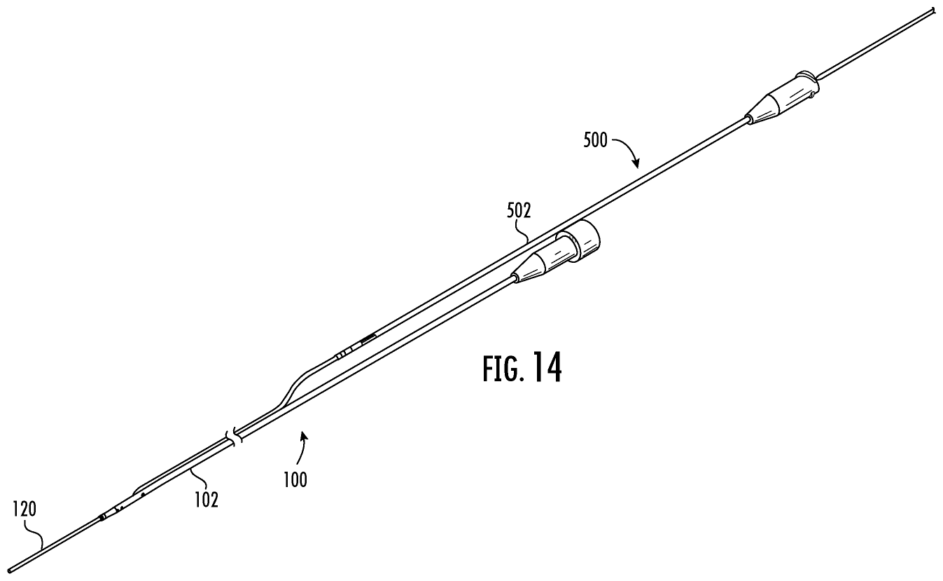

In some embodiments, a cannula 130, such as illustrated in FIG. 13, may be inserted into the lumen of a first tubular medical device such as any of the first tubular medical devices described herein. Like the stylet 110, the cannula 130 has an elongate body 132 extending between a distal end 131 and a proximal end 133 of the cannula 130. The distal end 131 of the cannula 130 is insertable into the lumen defined through the first tubular medical device, such as via a hub at the proximal end of the first tubular medical device. The proximal end 133 of the cannula 130 typically remains outside and proximal to the first tubular medical device, such as to be accessible to a medical professional. The cannula 130 may have a hub 134 at the proximal end 133 thereof, configured to be grasped by a medical professional, such as to guide the cannula 130. Additionally, the hub 134 may be configured to allow access to a lumen 136 extending through the cannula 130 between the distal end 131 and the proximal end 133 thereof. In some embodiments, the cannula 130 is more rigid than the stylet 110 described above and may therefore replace the stylet 110 in stiffening the tubular elongate body of a first tubular medical device such as described herein, such as to facilitate pushability of the first tubular medical device. Optionally, a stylet 110, such as described above, may be inserted into the lumen 136 of the cannula 130 to impart additional stiffness to the cannula 130 and thus to the first tubular medical device into which the cannula 130 and stylet 110 are inserted. Additionally, a guidewire may be guided through the distal opening 135 of the cannula 130 and proximally into the lumen 136 defined through the cannula 130 to allow the cannula 130 and the first tubular medical device to be guided over the guidewire to a target site within a patient. The distal end 131 of the cannula 130 may be advanced within the first tubular medical device to the distal end of the first tubular medical device to straighten a curved distal section of a first tubular medical device such as illustrated in FIGS. 9-12. If the distal end 131 of the cannula 130 is inserted to the distal end of the first tubular medical device, then the cannula 130 may include a side opening 137 spaced proximally from the distal opening 135 in the cannula 130 at substantially the same distance that the side opening in the first tubular medical device is spaced proximally from the distal opening thereof. As such, the position of the side opening 137 of the cannula 130 substantially matches/is aligned with the side opening of the first tubular medical device when the cannula 130 is inserted and assembled with the first tubular medical device. The guidewire may be extended through the distal end of the first tubular medical device and the distal end 131 of the cannula 130, and out the side opening of the first tubular medical device and the side opening 137 of the cannula 130. It will be appreciated that the guidewire may be withdrawn to allow the cannula 130 to be withdrawn to allow the curved distal section of the first tubular medical device to return to its pre-set curved configuration.

It will be appreciated that the second medical device may be a device which is substantially similar to the first tubular medical device. For instance, in the example of an embodiment of a system illustrated in FIG. 14, the first tubular medical device 100 and the second medical device 500 have respective tubular elongate bodies 102, 502 with a lumen 106, 506 defined therethrough, the tubular elongate bodies 102, 502 and lumens 106, 506 respectively extending between the distal end 101, 501 and the proximal end 103, 503 of the first tubular medical device 100 and the second medical device 500. The distal ends 101, 501 of the tubular elongate bodies 102, 502 may include curved distal sections such as described above with reference to the first tubular medical devices 100′, 100″ illustrated in FIGS. 9-12. Such optionally curved distal sections of the first tubular medical device 100 and the second medical device 500 are not illustrated as curved in FIG. 14 because the guidewire 120 extending therethrough, or an optional stylet or cannula, may straighten the distal sections of the first tubular medical device 100 and the second medical device 500. If the distal sections of the first tubular medical device 100 and the second medical device 500 are curved, withdrawal of the guidewire 120 (and optional stylet and/or cannula if provided) would allow the distal sections to return to preset curved configurations. As may be appreciated, because the second medical device 500 includes a side opening 507 through which the guidewire 120 exits and extends proximally, an additional medical device may be guided over the guidewire 120 and alongside the first tubular medical device 100 and the second medical device 500. Various further features of the first tubular medical device 100 and the second medical device 500 may be as described above with reference to the first tubular medical device 100 illustrated in FIG. 1 and FIG. 1A. The first tubular medical device 100 and the second medical device 500 may be used for aspirating and irrigating a target site to which the devices 100, 500 have been inserted. In some instances, the direction of fluid flow through the first tubular medical device 100 is opposite the direction of fluid flow through the second tubular medical device 500. For example, the first tubular medical device 100 may be used as the inlet lumen to a target site to flush and/or to pulse fluid to the target site, such as to stir up stone fragments/debris to be aspirated out with the second tubular medical device 500. However, other fluid flows as described above may be implemented.

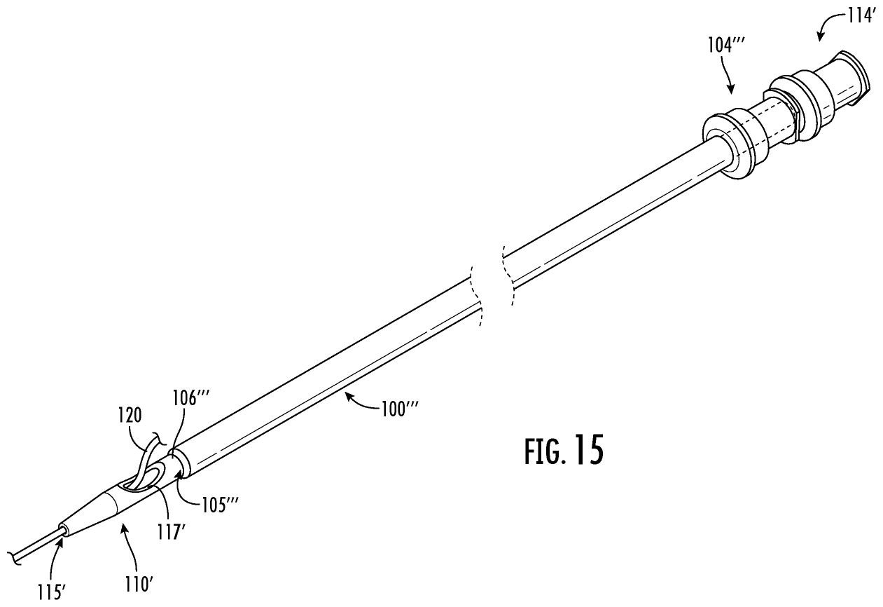

In accordance with various principles of the present disclosure, instead of the guidewire 120 extending through a side opening 107 through the wall of the first medical device, such as illustrated in FIG. 3, the guidewire 120 may extend through a side opening 117′ through a sidewall of a dilator 110′ extending through a first tubular medical device 100″', such as illustrated in FIG. 15. The guidewire 120 may be inserted through a distal opening 115′ of the dilator 110′ and extended proximally to extend out the side opening 117′ of the dilator 110′. As may be appreciated, the illustrated example of an embodiment of a dilator 110′ allows the guidewire 120 to extend alongside the first tubular medical device 100″' to provide a side rail over which a second medical device may be guided alongside and external to the first tubular medical device 100″', such as described above with reference to the first tubular medical device 100, 100′, 100″ of the various examples of embodiments illustrated in FIGS. 1-13. In some aspects, the first tubular medical device 100″' does not have a side opening, as in the above-described examples of embodiments, and only has a distal opening 105″' from which the dilator 110′ extends distally. The dilator 110′ and the first tubular medical device 100″' may be releasably connected by means of a slip fit, threads, etc., between the hub 104 of the first tubular medical device 100″' and the hub 114′ of the dilator 110′, such as in a manner known to those of ordinary skill in the art and thus not illustrated in detail in FIG. 15.

In accordance with various principles of the present disclosure, at least a portion (e.g., a distal portion) of the first tubular medical device 100″' may be formed into a pre-set, non-straight shape, such as described above with reference to the examples of embodiments illustrated in FIGS. 9-12. For the sake of brevity, reference is made to the above descriptions of the examples of embodiments of FIGS. 9-12 as being applicable to a first tubular medical device 100″' such as illustrated in FIG. 15. The pre-set curve of the first tubular medical device 100″' can be straightened by inserting the dilator 110′ through the lumen 106″' of the first tubular medical device 100″', such as from the proximal end to the distal end of the first tubular medical device 100″'. During the removal/withdrawal of the dilator 110′ from the first tubular medical device 100″', the non-straight (e.g., curved) section can be steered and/or positioned, even as the section is returning to its pre-set configuration.

It will be appreciated that various of the devices and systems described herein may be used for any of a variety of medical procedures, such as, without limitation, lithotripsy, as described above. In some aspects, lasers are used to fragment renal stone to very small sizes, such as less than 1 mm or to dust size (e.g., less than 250μ). Both a first tubular medical device and a second medical device formed in accordance with various principles of the present disclosure can be positioned with their respective distal ends in close proximity to the stone (e.g., at the lower pole calyx) using scope visualization and/or fluoroscope visualization. During lasing, the first tubular medical device can be used to aspirate stone dust, elevated temperature, and/or elevated pressure, while the second medical device (the ureteroscope) can be used to irrigate and flush fragments toward the first tubular medical device. For instance, use of the working channel of the ureteroscope for irrigation instead of for aspiration may reduce any possibility of stone fragments blocking/clogging the working channel if partially occupied by the laser fiber. Aspiration of stone dust may be performed after lasing, or in between lasing of more than one stone. The laser fiber may be removed from the ureteroscope's working channel, and the working channel may then be used to aspirate with the advantage of direct scope visualization. a front facing working/suction channel, and pull wire steerability to maneuver the scope even closer to the stone fragments. The first tubular medical device may be advantageously used for irrigation and/or to gently flush the stone dust towards the ureteroscope. If the medical professional prefers to aspirate only after or in between lasing, the first tubular medical device can be initially positioned further away from the fragments to avoid scattering the fragments with the irrigation flow.

A first tubular medical device with a preset non-straight (e.g., curved) distal portion may advantageously be used to access stones in both upper and lower poles of the kidney. Repositioning of lower pole stones (e.g., with a retrieval device) to the upper or mid pole of the kidney may allow easier access and/or tool placement, and may ease hand fatigue which otherwise may occur holding the deflection knob of the ureteroscope handle, and thereby may reduce the amount of time needed to perform the procedure.

In instances in which a reusable scope is used, the medical professional may want to reduce the potential risk of damaging the scope by aspirating fragments through the working channel thereof. In such instance, the first tubular medical device may be a catheter, and the second medical device inserted alongside the first tubular medical device, such as in a manner as described above, may be a steerable device such as a steerable guide catheter with no imager (no direct scope vision). Optionally, the first tubular medical device and the second medical device have pre-set curves, such as described above, with limited steerability and no imager (no direct vision). Aspiration of stone dust with the first tubular medical device and the second medical device may be performed after lasing substantially all of the renal stone(s) to dust using a scope with a laser fiber extended through the working channel of the scope. The scope and laser fiber are then exchanged with a first tubular medical device and a second medical device mounted in a manner such as described above. The first tubular medical device and the second medical device may be steered into position, over a common guidewire, such as described above. Fluoroscopy may be used to visualize the location of the devices and the irrigation/aspiration of stone fragments.

A first tubular medical device as described herein may be sold as a kit with one or more additional medical devices, such as one or more of the second medical devices described herein. The kit optionally includes a guidewire such as described above. At least the first tubular medical device, and one of the additional medical devices of the kit includes a lumen through which the guidewire is insertable, and configured to allow the associated medical device to be guided over the guidewire to a target site within a patient's body.

It will be appreciated that various features of one example of an embodiment of a medical device described herein may be present in others of the examples of embodiments described herein, whether or not explicitly stated or described. For purposes of clarity, not all components having the same reference number are numbered. It will further be appreciated that the various features described herein may be used singly or in any combination thereof. Therefore, the present invention is not limited to only the combination of features described with respect to the embodiments specifically described herein. Moreover, it will be appreciated that various operations of an above-described device or system may be performed by others of the above-described devices or systems.

Although embodiments of the present disclosure may be described with specific reference to medical devices and systems and procedures for treating the urinary tract, it should be appreciated that such medical devices and methods may be used to treat tissues of the gastrointestinal system, abdominal cavity, digestive system, urinary tract, reproductive tract, respiratory system, cardiovascular system, circulatory system, and the like. Moreover, the medical devices, instruments, tools, etc. of the present disclosure are not limited, and may include a variety of medical devices, instruments, tools, etc., for accessing body passageways, including, for example, ureteroscopes, duodenoscopes, catheters, bronchoscopes, colonoscopes, arthroscopes, cholangioscopes, cystoscopes, hysteroscopes, and the like. Such devices, instruments, tools, etc., may be disposable and for single use only, or sterilizable and reusable, the present disclosure not being limited in this regard.

Although embodiments of the present disclosure may be described with specific reference to medical devices and systems (e.g., endoscopic devices, accessory tools, and/or guidewires inserted near or through urinary tract, etc.), it should be appreciated that such medical devices and systems may be used in a variety of medical procedures for navigating one or more devices through ductal, luminal, vascular, or body lumen anatomies. The disclosed medical devices and systems may be inserted via different access points and approaches, e.g., percutaneously, endoscopically, laparoscopically, or combinations thereof, the present disclosure not being limited in this regard.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for accessing a target site within the body of a patient, said system comprising:
a first tubular medical device having a distal end, a proximal end, and a lumen defined therethrough between the distal end and the proximal end, said lumen configured to accept a guidewire to advance said first tubular medical device over the guidewire to the target site; and
a second medical device advanceable alongside said first tubular medical device over the guidewire said first tubular medical device is advanced over, the guidewire extending through said lumen defined through said first tubular medical device and a lumen defined through said second medical device.

2. The system of claim 1, wherein:
said first tubular medical device includes a tubular elongate body extending between the proximal end and the distal end of said first tubular medical device and a wall defining said lumen;
said tubular elongate body defines a distal opening at the distal end of said first tubular medical device;
said tubular elongate body defines a side opening through said wall thereof; and
a guidewire is extendable into the distal opening of said tubular elongate body, through a distal portion of said lumen therein, and out the side opening through said wall of said tubular elongate body to be accessible for extending into the lumen defined through said second medical device.

3. The system of claim 2, wherein said side opening is selected from one of the group consisting of a slot, a slit, a skive, a hole, or an aperture.

4. The system of claim 2, wherein said second medical device has a proximal end and a distal end, and includes a tubular elongate body extending between the proximal end and the distal end thereof having a wall defining a lumen configured to accept the portion of the guidewire extending out the side opening of said first tubular medical device.

5. The system of claim 4, wherein said tubular elongate body of said second medical device defines a distal opening at the distal end of said second medical device, said guidewire being extendable therethrough into the lumen defined through said second medical device.

6. The system of claim 5, wherein said second medical device is configured to allow the proximal end of the guidewire inserted therethrough to exit said system through the proximal end of said second medical device.

7. The system of claim 2, further comprising a cannula having a distal end and a proximal end, wherein:
said cannula is configured to be insertable through said lumen defined through said first tubular medical device;
said cannula has a wall defining a lumen therethrough extending between the distal end and the proximal end of said cannula;
a distal opening is defined in the distal end of said cannula in communication with said lumen through said cannula;
a side opening is defined in said cannula wall in communication with said lumen of said cannula; and
the distance between the distal opening and the side opening of said tubular elongate body of said first tubular medical device is substantially the same as the distance between the distal opening and the side opening of said cannula.

8. The system of claim 1, wherein said first tubular medical device is a sheath.

9. The system of claim 8, further comprising a cannula insertable through said lumen defined through said first tubular medical device.

10. The system of claim 9, further comprising a stylet insertable through a lumen defined through said cannula.

11. The system of claim 7, further comprising a stylet insertable through said lumen defined through said first tubular medical device.

12. The system of claim 1, wherein said second medical device comprises a device selected from the group consisting of: a scope, a flexible scope, a steerable catheter, a guide catheter, a non-steerable catheter, a sheath, or an access sheath.

13. The system of claim 1, wherein said first tubular medical device includes a curved distal section formed of a shape memory material and capable of being straightened for insertion to the target site within the patient.

14. The system of claim 1, wherein said second medical device is steerable and capable of steering said first tubular medical device when said second medical device and said first tubular medical device are extended over a common guidewire.

15. A kit for accessing a target site within the body of a patient, said kit comprising:
a guidewire;
a first tubular medical device having a distal end, a proximal end, and a lumen defined therethrough between the distal end and the proximal end, the lumen configured to accept said guidewire and to be advanced over said guidewire to the target site; and
a second medical device advanceable over said guidewire alongside said first tubular medical device with said guidewire extending through the lumen defined through said first tubular medical device and a lumen defined through said second medical device.

16. The kit of claim 15, wherein:
said first tubular medical device includes a tubular elongate body extending between the proximal end and the distal end of said first tubular medical device and defining said lumen;
said tubular elongate body defines a distal opening at the distal end of said first tubular medical device;
said tubular elongate body defines a side opening through the wall defining said lumen; and
said guidewire is extendable into the distal opening of said tubular elongate body, through a distal portion of said lumen therein, and out the side opening through the wall of said tubular elongate body to be accessible for extending into the lumen defined through said second medical device.

17. The kit of claim 15, wherein said second medical device comprises a device selected from the group consisting of: a scope, a steerable catheter, a guide catheter, a non-steerable catheter, a sheath, or an access sheath.

18. A method of accessing a target site within a patient, said method comprising:
guiding a first tubular medical device over a guidewire; and
guiding a second medical device over the same guidewire the first tubular medical device is guided and alongside the first tubular medical device.

19. The method of claim 18, further comprising extending a proximal end of the guidewire into a distal end of the first tubular medical device and into a lumen extending therethrough, and out a side opening through a wall defining the lumen.

20. The method of claim 19, further comprising extending the proximal end of the guidewire into a distal end of the second medical device and into a lumen defined through the second medical device to guide the second medical device over the guidewire alongside the first tubular medical device.
